(19) United States
(12) Patent Application Publication
(10) Pub. No.: US 2020/0377838 A1
(43) Pub. Date: Dec. 3, 2020

(54) A Microfluidic Device for Culturing Cells Comprising A Biowall, A Bead Bed and A Biointerface and Methods of Modelling Said Biointerface Thereof

(71) Applicant: National Research Council of Canada, Ottawa (CA)

(72) Inventors: Teodor VERES, Montreal (CA); Xuyen Dai HOA, Montreal (CA); Jamal DAOUD, Montreal (CA); Caroline MIVILLE-GODIN, Montreal (CA); Lidija MALIC, Montreal (CA)

(73) Assignee: National Research Council of Canada, Ottawa, ON (CA)

(21) Appl. No.: 16/994,144

(22) PCT Filed: Feb. 18, 2019
(86) PCT No.: PCT/IB2019/051298

§ 371 Date: Aug. 14, 2020

Related U.S. Application Data
(60) Provisional application No. 62/631,977, filed on Feb. 19, 2018.

(51) Int. Cl. C12M 1/12; C12M 3/06; C12M 1/00; B01L 3/00

(52) U.S. Cl. CPC C12M 25/18; C12M 23/16; C12M 23/20; C12M 29/04; B01L 2200/0689; B01L 3/502761; B01L 2200/0652; B01L 2300/0877; C12M 23/38

(57) ABSTRACT

A technique for producing an artificial biointerface involves providing a patterned microfluidic chip having: a chamber divided by a fluid-permeable fencing into a central region and two flanking channels; and at least 3 fluid paths, each of the paths extending across one of the central region and the two flanking channels. A porous packing of rigid beads is placed within the central region to define a bead bed, the beads being of a size to be retained by the fencing. A biowall can be grown on at least one segment of the fencing separating the central region from one flanking channel, the biowall formed at least in part by live cells cultured on the beads. Beads may be modified, coated or functionalized to improve cell attachment and growth, and for reporting, or dosing particles or molecules can be conveniently added to the bead bed.

### FIELD OF THE INVENTION

The present invention relates in general to a technique for supporting cells to form and sustain biological tissues so that the tissues model a biointerface and, in particular, to such a support in the form of a porous bed or packing of rigid beads with controlled porosity, as well as a microfluidic device incorporating this support.

### BACKGROUND OF THE INVENTION

Organs on Chip (OoCs) are now intensively researched models that leverage advances in microfluidic device platforms and tissue engineering to emulate functioning of some aspects of organs, tissues, and mechanisms within animals, and possibly plants. Important biointerfaces such as cellular barriers, organ boundaries, or host barrier interfaces all have respective roles to play in furthering the advance of understanding biology, pharmacology, immunology and medicine. Biointerfaces, as the term is used herein, include an engineered or excised tissue or organ, or cultured tissue, including at least one biowall (the term used herein to distinguish walls of a tissue or organ from microfluidic structures) comprising interconnected cells forming a tissue barrier dividing an interior of the model tissue or organ (e.g. a placenta, brain, gland), from an inter-region surrounding the biowall. A second biowall may face the inter-region from a side opposite the biowall to form a two-biowall biointerface, or the inter-region may be a mucosa facing a non-sterile environment, for example. Thus the inter-region opposite the biowall may be a liquid (e.g. kidney, placenta, blood brain barrier) or gaseous (lung, esophagus, stomach) environment. The biointerface defines a formal cellular interface supporting complex exchanges of signaling molecules, nutrients, and waste products. Even the modeling of cells bordering mineralized tissues (tooth or skeletal tissues) will typically involve a gelatinous or liquid interface region. For example, such interfaces can involve co-cultures of osteoblasts/osteoclasts with endothelial cells or myelinated/unmyelinated neurons for analysis of nutrient transfer or sensory activation, respectively.

One application of OoC biointerfaces is to guide drug development with intelligent, low cost, tests[1] on candidate drugs prior to drug screening. With drug development typically taking 10-15 years before approval, the overall success rate being low[2], and the costs of screening new drugs having risen dramatically over the past 10 years[3]; low

cost, prescreening tests are sought to improve the selection of drug candidates for screening. The more accurately physiological conditions can be modelled by the test, the better confidence drug developers will have in their decisions to rank candidate drugs for screening. OoC biointerfaces offer clear advantages in this respect.

Apart from the potential to improve drug development pipelines, other applications include testing of chemical toxicity, as well as the study of cellular processes, tissue response, organ functions, etc. As far as OoCs biointerfaces offer more realistic in vitro human models, and lower cost, animal friendly, and/or more human centered alternatives to in vivo animal models, OoC biointerfaces will remain attractive platforms for studies.

Microfluidics and micro-technology offer excellent tools for constructing organ or tissue-on-a-chip applications. The ability to fabricate at the length scale of cells (10-100 μm) and to manipulate particles and fluidics with precision at low flow rates, is essential to creating an environment for the culture and sustenance of cell tissues. Microscopic flow control and microstructuration are important design capabilities leveraged for OoC applications. The literature presents a number of OoC demonstrations [4]. Various obstacles have been overcome in terms of material compatibility, cell alimentation and growth, and maintaining cell nature in the artificial environment.

Material compatibility with cell growth has been a perennial problem, as few plastics naturally support cell growth without treatments and structuration. The processes for treating plastic microfluidic chips to permit cell growth remain expensive, time consuming, time sensitive, error prone, and/or inconvenient in general. For example, it is known that some oxygen plasma treatments that promote activation of surfaces of polymers (required for many treatments), make it difficult to subsequently bond the polymer surfaces to seal microfluidic channels, and polymeric structures that allow for cell growth of one tissue, does not necessarily allow for growth of other cells.

The development of biointerfaces requires a scaffold or support for cell growth of at least one biowall. The scaffold is provided to anchor cells, and to permit supply of nutrients and egress of waste. It is commonplace to use a sheet of collagen, such as a vitrified collagen sheet to form this scaffold, as this is highly biomimetic for several tissue environments. For example, a paper to Ji Soo Lee et al. entitled “Placenta-on-a-chip: a novel platform to study the biology of the human placenta”, teaches a collagen membrane formed by gelation and vitrification of collagen separating an upper microfluidic channel from a lower microfluidic channel on adjacent patterned PDMS films, where the microfluidic channels overlap. The collagen membrane permits independent fluidic access to the upper and lower channels. The upper and lower channels were coupled to respective cell culture media reservoirs via tubes and respective cell cultures (a co-culture) were grown on each side of the collagen membrane, although the collagen membrane is eventually infused with cells of both type almost homogeneously distributed.

The ingrowth and migration of the cells illustrates a possible problem with some biointerface models that require a regular separation of co-cultured cells to form parallel facing first and second facing biowalls, or a minimum thickness of mucosa that is not natively controlled by the tissue. Thin glassy collagen membranes may not be satisfactory for this purpose.

It is also known to use hydrogel structures as scaffolds for supporting cell growth, as hydrogels do allow for fluid transport and dispersal of molecular species carried in the aqueous fraction of the hydrogel. U.S. Pat. No. 9,231,496 to Kamm et al. teach a microfluidic device with one or more gel cage regions, each of which flanked by one or more fluid channels to create gel cage region-fluid channel interfaces. Gel is contained by a porous wall consisting of pillars having properties for retaining the hydrogel. It is noted that the gel cage regions are not addressable to microfluidic channels, except via these interfaces which offer a relatively small surface area for interacting with the hydrogel and a back-side of the biowalls. The only way to reach the backsides of the biowalls are via gel inlet ports, which is unfortunate because separate and distinct alimentation and waste channels, or delivery channels, cannot be provided to the interface region any other way.

US 2015/0377861 to Pant et al. teaches a cell culture assay device for high throughput cell-based assays with increased physiological fidelity. The device of FIG. 8 (see [0061]) includes microfluidic walls 115 separating tissue space 13 surrounded by linear flow channels 114. The walls 115 are permeable to aqueous buffers and formed by plastic structures 115b that are separated by gaps 115a (0.2-5 μm), although these walls may alternatively be porous walls with 0.2-30 μm porosity. At para. [0065] it is stated that the “channels forming SMNs, IMNs, bifurcations, and the luminal surfaces of the tissue spaces may be coated with” a variety of “molecules to assay for associations with particles or to facilitate grown of cells”. The list of materials include known hydrogel scaffolds for cells, and “alginate beads”. Alginate beads are gel beads that are known for encapsulating materials and, as such, generally have liquid cores, which agrees with all of the gel materials in the list. These channels may reasonably be understood to be limited to those inside the tissue spaces 13 where the cell growth is intended. There is no specific suggestion of a gel bead surrounding the “linear flow channels 114” according to these teachings. A “monolayer” of cells to be grown will be edge-connected to the interface 114, as explained at [0062], and no biointerface is produced or suggested to be modelled, by Pant et al.

In a non-analogous field of bulk production of cell-secreted products, where non-microfluidic solutions are sought to scale up animal cell propagation, it is known, for example from U.S. Pat. No. 5,102,790 to Bliem et al., to grow animal cells (including anchorage independent cells that are generally incidental, or unrelated, to biointerface formatting) in packed beds of carrier particles, such as glass beads. The purpose for selecting bead beds is to increase a number of niches for the animal cells, while still facilitating regular alimentation across the whole area to support all cells.

It is by no means clear having regard to these teachings that in a microfluidic environment, where dimensions and fluid distribution mechanisms are substantially different, and where the cells to be cultured are particular to biointerfaces, whether glass beadbeds would produce biowalls required to serve as models of biointerfaces.

Thus, while there are microfluidic devices with porous walls and structures for supporting and alimenting cell cultures, there are no structures taught for supporting cell cultures that provide separate fluidic access to more than front and back faces of cell cultures, and the cage region-fluid channel interfaces of Kamm et al. offer very limited surface area for interacting with the hydrogel. Accordingly there is a need for an improved cell culture support structure, especially one that improves interaction with the cell cultures, and especially in between co-cultures.

### SUMMARY OF THE INVENTION

Applicant has discovered that a packing of rigid beads (bead bed) will allow for the growth of distinct cell cultures to form one or more biowalls at or near a surface of the packing. Rigid beads have numerous advantages over gels and collagen networks in this role:
the rigid beads prior to packing may be coated or treated ex situ to facilitate cell growth, which overcomes problems with coating plastic microchannels;
multiple subsets of the rigid beads may have different respective compositions, morphologies (size, surface textures and shape: spherical, half-shell, rods, cubes, star shaped, etc.), surface treatments or coatings, to collectively provide better cell growth and adherence than any single kind of rigid bead, unlike microfluidic walls;
each subset of the rigid beads can be separately functionalized, in batches, can be mixed in proportions and injected with concentration of beads of different concentrations, for discrete phases or concentration gradients;
packings of rigid beads easily form reliable bed thicknesses, and cell scaffold structures for separating two biowalls or defining or supporting a mucosa, and can be arranged to follow any curves or contours appropriate for the organ or tissue model, by design of a microfluidic chip supporting the bead bed;
this separation provides an expanded region through which alimentation of an inter-region or mucosa is possible, distinct from alimentation to the internal side of the biowall, or through a second biowall;
bead beds allow for incorporation of separate reporter, sensor, or delivery beads, particles, or objects with reliable fixity and with less cost and effort, which allow these beads, particles, or objects in situ access to the inter-region;
such beads, particles, or objects, or coatings of beads may provide controlled release, e.g. selective release depending on triggers (pH, chemical, thermal, pressure, ultrasonic, photo, electric, or magnetic) either sensed in situ within the inter-region, or externally driven, or with time; and
the beads, particles, or objects, or coatings of beads may interact with the alimentation streams to: prompt emission of signaling entities into a waste stream, to absorb or catalyse reactions; or to biodegrade, bioresorb, or decompose;
the interaction may promote or inhibit reporting, sensing or chemical release; and
monitoring waste products can provide feedback for varying alimentation of a biowall, inter-region, mucosa, or environment.

The use of bead beds to create a scaffold having a shape, porosity, and surface structure for supporting a viable barrier between cell subpopulations is demonstrated herein below. Leveraging these advantages allows for the integration of functionalities through controlled perfusion and space- and time-localized surface modification, to permit viable cell (tissue) co-culture while simultaneously assaying cell-cell communication in response to induced biochemical stimuli. For example, multi-phase bead beds of sequential populations of antibody-modified beads targeting various biomarkers for cell-cell communication can be useful. Furthermore a controlled phase of the bead bed can be used as a calibration phase for in situ detection of released markers and time evolution of the biointerface. Each of the one or more biowalls can have a respective culture or may include two or more cell lines, adjacent to, and supported by, the aforementioned bead bed. Such a system with alimentation to an interface region and non-facing sides of the biowalls, or to the non-facing side of the biowall and facing the mucosa, allow for a model that can advance biointerface studies of physiologically relevant cell interactions. The biointerface model can include both biomolecule capture at the interface, as well as controlled release of biological factors via the separation bead bed barrier, to further elucidate and artificially stimulate cell-cell signaling.

Accordingly, a method is provided for modelling a biointerface, the method involves: providing a microfluidic chip, the chip patterned to define a chamber divided into a central region and a first and second flanking channels that flank the central region, the division provided by a fluid-permeable fencing; and at least 3 microfluidic ports, including at least two ports located at opposite ends of the chamber and at least one port in each of the central region and two flanking channels; localizing a porous packing of rigid beads within the central region to define a bead bed, the beads having a mean size, between 2 and 300 μm, sufficient for the fencing to retain the beads while fluid permeates the fencing; and growing a biowall on at least one segment of the fencing separating the central region from one flanking channel, the biowall formed at least in part by live cells cultured on the beads, by alimenting cells through the pairs of microfluid ports. Preferably each of the central region and a first and second flanking channels has two ports at opposite ends of the chamber.

The method may further comprise: coating the chamber with a cell adhesion promoting coating; localizing the porous packing by introducing a mixture of the beads into the chamber; seeding at least one cell culture through at least one of the flanking channels; and incubating while alimenting the at least one cell culture.

Introducing the mixture of the beads into the chamber may involve: injecting the bead mixture in a liquid carrier through one of the ports of the central region while extracting fluid at one of the other ports; or placing a pressed mixture of the beads into the central region with a cover of the microfluidic chip removed. Introducing the mixture of the beads into the chamber may involve introducing at least two phases into respective parts of the central region, each phase having a different constituency in terms of at least one of: a mean size, mean shape, surface texture, functionalization, composition, or coating of the beads, or fractional populations of a mixture of such beads along with any non-rigid beads, particles or objects. The respective parts of the central region may partition the central region in strata parallel to the fencing, or in lines perpendicular to a flow between a pair of ports of the central region. These respective parts may be separated by additional fencing if additional ports are provided to respective parts of the central region.

The central region is preferably an elongated flow path through the patterned microfluidic chip having a length between two opposing ports that is at least 2 orders of magnitude greater than an etch depth dimension of the central region.

A kit for producing an artificial biointerface is also provided. The kit includes: a patterned microfluidic surface, the pattern defining a chamber divided into a central region and a first and second flanking channels that flank the central region, the division provided by a fluid-permeable fencing; a source of rigid beads adapted to form a bead bed within the central region, the beads having a mean size between 2 and 300 μm, sufficient for the fencing to retain the beads while fluid permeates the fencing; and a cover for the microfluidic surface, the cover dimensioned for enclosing the chamber and adapted to seal the chamber from ambience; wherein at least one of the patterned microfluidic surface, and cover provide at least 3 microfluidic ports, with two of the ports at opposite ends of the chamber, and at least one port in each of the central region and the two flanking channels. Preferably each of the central region and the two flanking channels has at least two ports at opposite ends of the chamber.

The kit may be assembled with the bead bed formed within the central region and the cover enclosing and sealing the chamber. The assembled kit may have a biowall formed along a segment of the fencing separating the central region from one flanking channel, the biowall formed at least in part by live cells cultured on the beads, the biowall being alimented by the microfluidic ports.

Furthermore, in accordance with the present invention an artificial biointerface is provided. The biointerface includes a microfluidic chamber divided into a central region and a first and second flanking channels that flank the central region, the division provided by a fencing, where the central region filled with a porous packing of rigid beads. The beads have a mean size between 2 and 300 μm, sufficient for the fencing to retain the beads while fluid permeates the fencing. The biointerface also includes a biowall located along a segment of the fencing separating the central region from one flanking channel. The biowall is formed at least in part by live cells cultured on the beads. At least 3 microfluidic fluid paths are provided, each of the paths extending across one of the two flanking channels, and the central region, for supplying and extracting fluid from the respective flanking channel or central region.

The beads may be composed of a polymer, silica, metal, or ceramic; preferably of a styrenic polymer or silica, and may be treated to: improve cell adhesion or growth; to selectively bind to a target molecule or particle; to report binding to a target molecule or particle; or to selectively release a molecule or particle. Binding, release, or report of binding of the target molecule or particle may be time dependent, or in response to optical, thermal, electrical, magnetic, chemical (including pH), or mechanical (including ultrasonic) stimulation. The packing of rigid beads may include at most 25% of non-rigid beads, particles or objects, or preferably at most 20%, 15%, or 12%, or 10% or 7% or 5%. The packing of rigid beads may include two or more phases, each phase having a different constituency in that terms of at least one of: a mean size, mean shape, surface texture, functionalization, composition, or coating of the beads, or fractional populations of a mixture of such beads along with any non-rigid beads, particles or objects.

The fencing may have through-holes from the flanking channel side to the central region that are smaller than a diameter of the smallest 10% of the beads. The fencing may have a 1 D, 2D or 3D curvature for delimiting the packing of beads to define a shape suited to mimicking a geometry of a natural tissue.

Further features of the invention will be described or will become apparent in the course of the following detailed description. An exact copy of the claims is incorporated herein by reference.

### BRIEF DESCRIPTION OF THE DRAWINGS

In order that the invention may be more clearly understood, embodiments thereof will now be described in detail by way of example, with reference to the accompanying drawings, in which:

FIG. 1 is a schematic illustration of a patterned film for defining principal components of a microfluidic device for producing a substrate for culturing or co-culturing cells in accordance with a strip channel embodiment of the present invention;

FIG. 1A is a schematic illustration of a variant of the patterned film of FIG. 1 showing a divided central area, and chevron flow control features in one division;

FIG. 1B is a schematic illustration of a variant of the patterned film of FIG. 1 showing a U-shaped central area, and an impermeable wall within an interior flanking channel;

FIG. 1C is a schematic partial illustration of a variant of the patterned film of FIG. 1, in which active valves permit improved fluid control within flanking channel;

FIG. 1D are schematic illustrations of top and bottom patterns of a patterned film for assembly with a second instance of the film, and a bead bed, to form a substrate for an equiaxed artificial biointerface in accordance with an embodiment of the invention;

FIG. 2A is a schematic illustration of a method for producing an artificial biointerface in accordance with an embodiment of the present invention, by injection through ports;

FIG. 2B is a schematic illustration of a method for producing an artificial biointerface in accordance with an embodiment of the present invention, with placement of a bead bed;

FIG. 3 is a schematic illustration of the patterned film of FIG. 1 with a bead bed packing and monoculture biowalls along fencing to both flanking channels;

FIG. 4 is a panel showing A a schematic illustration of the chip pattern bearing a divided chamber, with an enlargement of the chamber; B an image of a chip bearing this pattern produced to demonstrate the present invention; and C the chip with 6 pressure control lines between 3 pairs of ports;

FIG. 5 is a panel of 4 micrograph images at respective enlargements, showing A the whole chamber and leads to the ports; B the whole chamber; C the fencing at a downstream end of the chamber; and a close up view of the fencing;

FIG. 6A is a schematic illustration of a set up used for incubation and imaging of the biowall during its growth and culturing;

FIG. 6B is a photograph of a set up used for incubation and imaging of the biowall during its growth and culturing;

FIG. 7 is a panel of three images of an experiment used to determine optimal flow rates during seeding;

FIG. 8A,B are micrograph images showing cell seeding procedures for first side (8A), and two-sided cell seeding (8B);

FIG. 9 is a sequence time-lapse micrograph images showing cell wall growth, and a viable culture produced.

### DESCRIPTION OF PREFERRED EMBODIMENTS

Herein, an artificial biointerface is disclosed, along with a method for modelling a biointerface, and a kit, that allow for separate alimentation of a tissue region, inter-region and environment or second tissue region. The artificial biointerface may be provided on a microfluidic device. The microfluidic device includes a microfluidic chamber with at least one fluid-permeable fencing that divides the chamber into at least 3 volumes. At least one of these volumes contains a porous bed of (at least 75 vol. %, more preferably at least 80%, 85%, 87%, 90%, 97%, 95%) rigid particles (beads). At least one peripheral surface of the porous bed provides a scaffold for cell culture, and (at least) 3 microfluidic paths are defined for fluids: one for each of the 3 volumes. The artificial biointerface further comprises a biowall of a tissue grown on the scaffold.

As is well known in the art, a convenient route for forming microfluidic devices is to produce a relief pattern on a foil or film, and bonding a layer of over the top of this relief pattern to enclose the patterned surface, whereby recessed areas of the relief pattern become channels, cavities and openings for microfluid contents, and the pattern dictates interconnection of these channels and cavities.

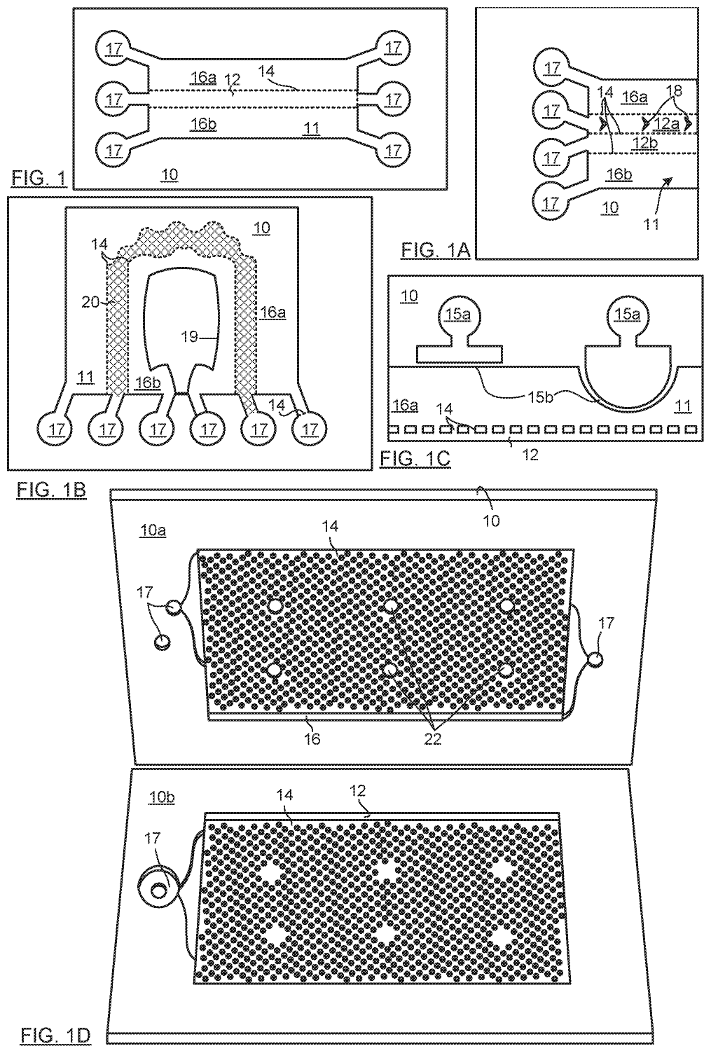

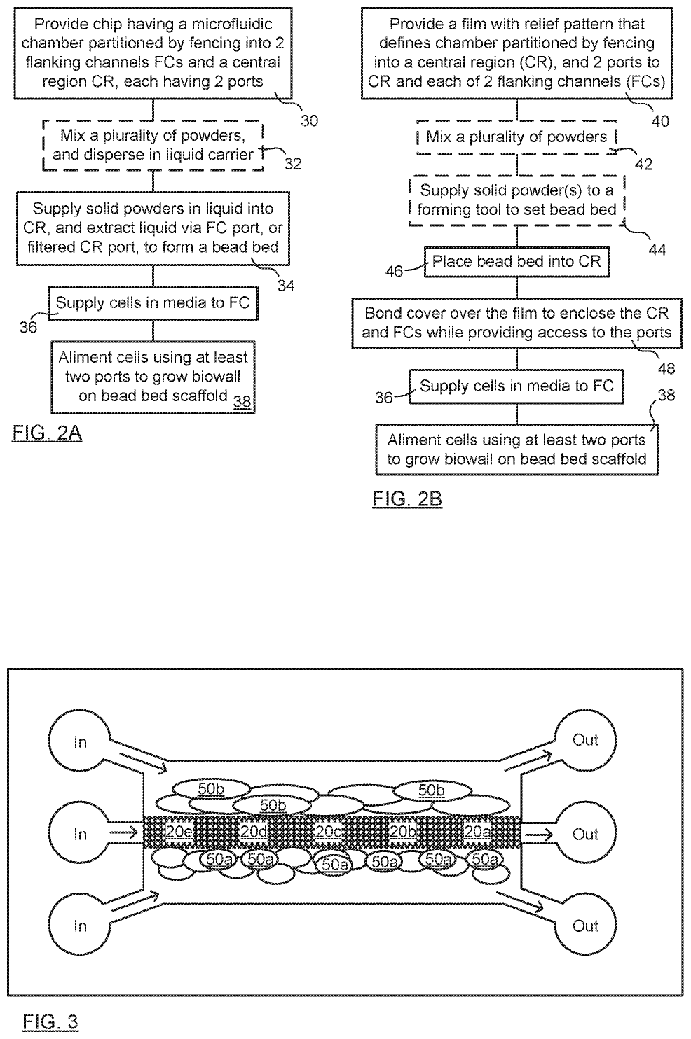

FIG. 1 is a schematic plan view of a pattern for a film 10 for use in forming an artificial biointerface in accordance with an embodiment of the present invention. The pattern in film 10 defines a recessed chamber 11 for the artificial biointerface. Film 10 may be formed of substantially any suitable material. Particularly preferred are materials of non-reactive plastic, metal, ceramics, glass, and combinations thereof, that are permanently patterned with low cost processes, readily bonded with a flat cover surfaces, and forming fluid-tight seals with minimal pressure, temperature and time, even with surfaces that are not highly flat, or having well matched contours. Further advantageous materials are gas permeable, or selectively gas permeable. For example, the film 10 may be composed of: a biocompatible polymer such as: a siloxane based elastomer, a mixture of siloxane based elastomers, a thermoplastic elastomer TPE (preferably an oil free styrenic block co-polymer), a mixture of TPEs, a mixture of one or more TPEs with one or more hard thermoplastic phases, cyclic olefin copolymer, or polytetrafluoroethyl-ene; glasses such as fused silica or quartz glass; ceramics such as titania (i.e. titanium dioxide); or atomically thin semiconductors such as Transition Metal Dichalcogenide (TMD), boron nitride, or graphene. Combinations of metals, ceramics and glasses within biocompatible polymers may also be applicable for example by doping or embedding particles or surface treating a biocompatible or non-biocompatible polymer. Patterning depth is preferably at least 2-3 times a mean diameter of the cells to be cultured (e.g. 20 to 500 μm), and a thickness of the film 10 is preferably 1.2-10 times the pattern depth.

A central region 12 of the chamber 11 is shown surrounded on 3 sides by fencing 14, but only two longitudinal fences are required to divide the chamber 11. The fencing 14 at the end of the chamber 11 is useful for controlling bead bed deposition if introduced fluidically. The fencing 14 is permeable to aqueous buffer, solvents, cell media and entrained gaseous micro bubbles (e.g. CO₂ and O₂), but retains a packing material consisting of rigid beads (herein ‘bead bed’). The central region 12 has a low surface area to perimeter ratio, such as is provided with a length from more than 5 times to more than 200 times the width or height (defined by etch depth), as in the rectangular central region 12 shown.

The fencing 14 separates the chamber 11 into two flanking channels 16a,b and a central region (CR) 12, in the form of a strip. Each of the flanking channels 16a,b and the CR 12 has a respective set of two fluid ports 17 (inlets/outlets). The fluid ports 17 of the flanking channels 16a,b are reversible (an inlet at one point in a process can be an outlet the next), but if the beads are loaded into CR 12 through the ports 17, and to prevent fluid pressure from entraining beads (this may be required depending on how loosely the beads are held in the bed to avoid eroding the bead bed), it may be preferable to maintain unidirectional flow through CR 12 (from left to right as shown). Alternatively filters may be coupled to the ports 17 of CR 12 after the bead bed is set, making the ports bidirectional. While three pairs of ports, each at opposite ends of the chamber, are shown, it will be appreciated that no illustrated process requires all six.

It will be appreciated that while the fencing 14 is illustrated as a single connected fence, it is equivalent to 3 fence segments, one separating the CR 12 and flanking channel 16a, one separating CR 12 and flanking channel 16b, and one marking an end of CR 12. The end fence 14 may equally be provided within communication lines between CR 12 and outlet port 17 (right), and may be removable as a porous plug, for example. As each fence segment of the fencing 14 is provided to retain a same bead bed, it may have a common porosity, composition and structure, however if a biowall intended for one cell culture has a particular preference for cell growth, or a need for higher hydrodynamic resistance than the other fencing, each fencing segment can be provided accordingly. If anchor cell integration with the bead bed is required to different degrees, or sizes of cells are different, it can be advantageous to tailor both the bead bed and possibly fencing.

The fencing 14 is part of the relief pattern applied to the film 10, and may consist of a track of full depth pillars. For example, each pillar may have a same cross-section, a uniform profile from base to top, which may be substantially a rectangular base cross-section with a tapered cross-section (monotonically decreasing length and width as a function of elevation from the base, because tapered cross-sections may be more easily and reliably formed, though any other shape that is convenient for forming, sufficient for retaining the bead bed, and sufficiently permeable, can be used alternatively. Preferably, elements of the fencing 14 include gaps having a mean pore equivalent diameter of 0.1-1000 μm, more preferably 0.5-200 μm, and most preferably from 0.5-50 μm. The fencing gap size will dictate a bead diameter for which the patterned film will be used, in that the mean bead diameter is preferably at least 5% larger than the pore diameter, although other differences may be preferred depending on the range of sizes of the beads, aspect ratios of the particles and fencing gaps, etc., as these may be sufficient for retaining these beads. Preferably a diameter the beads is a distribution with the smallest 10% having a diameter bigger than the equivalent diameter of the pores or gaps, especially if the insertion method is microfluidic. In some applications, the fencing gaps are chosen to ensure a hydrodynamic resistance across the fencing 14 that is significantly lower than that of a packing of such beads (i.e. a bead bed). Preferably the mean pore size is larger than a smallest dimension of the cells, for intercalation of the cells within the bead bed.

FIG. 1A schematically illustrates part of a first variant of FIG. 1. Herein like features of different variants are identified by the same reference numbers, and their descriptions are not repeated: they are only further explained to show a difference between the variants. Each variation is an independent feature and each subset of the variations is a corresponding embodiment of the present invention.

While FIG. 1 illustrates an undivided CR 12, it will be appreciated that a divided CR may be desirable, in order to distribute beads of respective functionalizations, sizes, morphology, or other properties in a controlled manner. A divided CR 12 may be desirable even if a homogeneous bead bed is desired, for example to control delivery to one side of the CR, and may also be advantageous if timed pressure variations at the 4 ports 17 allow for circulation of delivered fluid in a more continuous and better distributed manner. A core and shell structure, or layered bead bed may be set by providing additional fencing 14 and additional ports 17 between fenced regions. The variant of FIG. 1A is a partial view of a patterned film 10 featuring a CR divided in two parts 12a,b, which are separated by a fencing 14. Separation of the CRs 12a,b allows for forming of two adjacent bead beds where each bead bed can respectively receive beads of different (mixture of) size, morphologies, densities, surface treatments, or other functional properties. Naturally three or more bead beds may be formed. The fencing 14 that divides the CR may have a same porosity, composition, or structure as the fencing 14 that surrounds the CR, or may provide a higher hydrodynamic barrier.

While FIG. 1 shows a smooth bottom surface of the chamber 11, it may be advantageous for uniform packing of beads into the CR 12 (or a part thereof) to encourage a more turbulent flow of the supplied beads in a carrier liquid. If a mixture of beads are provided, that have various mass densities, sizes, or morphologies/fluid resistances, turbulence can improve homogeneity of the bead bed. Chevron features 18 are illustrated in FIG. 1A as a flow control device in part 12a of the CR. Chevron features 18 are particularly useful for encouraging turbulent flows, which can improve randomization of the packing of bead beds. While turbulence can be advantageous for certain packing arrangements, it will be appreciated that the natural settling and sorting of particles in laminar flow can be engineered to distribute particles in a desired arrangement. Flow control elements similar to the chevrons 18 can alternatively be used to increase sorting of the particles during flow. Chevrons 18 and other flow control features can be small relief height features or can extend the full depth of the part 12a, for example from 0.5 to 1 times the depth of the chamber 11.

While FIG. 1 shows a linear arrangement with two flanking channels 16a,b of equal dimension, it will be appreciated that a wide variety of structural shapes can be provided for the CR 12 and for the flanking channels 16a,b. FIG. 1B schematically illustrates a variant with a generally U shaped CR 12, with an external flanking channel 16a, and an internal flanking channel 16b. Each embodiment of the present invention generally provides the CR defining an elongated flow path between ports thereof, with the length between these ports being greater by at least 2 orders of magnitude than the other two (mean) dimensions.

While FIG. 1 shows two flanking channels 16a,b of equal dimension, it will be appreciated that a wide variety of structural shapes can be provided for the CR 12 and for the flanking channels 16a,b. While the embodiment of FIG. 1 shows a strip with parallel fencing 14 defining the flanking channels 16a,b, it will be appreciated that some biointerfaces require variability provided by a torturous path between opposing biowalls, or a mucosal support that has varying thickness at various points along the periphery. The variant shown in FIG. 1B shows a schematic example of such a torturous path with varying separations along one side.

FIG. 1B also shows a variation that involves a flow control feature within the internal flanking channel 16b, in the form of an impervious wall 19 that directs flow away from a central space of the flanking channel 16b, and encourages flow along a periphery of the flanking channel 16b, where it meets the fencing 14. Unlike the other figures, the CR is shown filled with a bead bed 20. A fence segment provided within the CR is provided at a fluid connection to port 17.

FIG. 1C illustrates a variant that incorporates valves within the flanking channel 16a. Two valves 15a are shown, one in an extended position, and the other in a contracted pose. Side valving by providing through-bores of a soft TPE is known in the art, and is taught, for example, in Applicant's WO2017066869 and U.S. Pat. No. 9,435,490. Variable actuation pressures result in the valves blocking the flanking channel 16a to various extents, and these valves can be used to vary flow through the flanking channel and interaction with segments of the fencing (or biowall adjacent thereto once cultured).

While the embodiment of FIG. 1 provides the fencing 14 between the CR 12 and flanking channels 16a,b limited by a pattern depth in one direction, this may not provide sufficient surface area for a desired biowall, or may not provide a desired aspect ratio of the artificial biointerface, or surface area to perimeter ratio. FIG. 1D shows top and bottom sides of patterned film 10 in accordance with a further variant of the embodiment of FIG. 1. In FIG. 1D, one side of film 10a provides a flanking channel 16 as a recess from the surface. The recess terminates in a porous fence 14. The fence 14 is preferably provided in the form of a micro, or nanoporous membrane, and may be formed, for example, as taught in Applicants U.S. Pat. No. 9,498,914, or in WO 2017/066869. Alternatively a hard TPE membrane can be provided, cut to size, and bonded to a soft TPE frame that was already patterned, or is patterned after the bonding. The frame may partially cover top and bottom surfaces of the membrane, but leaves at least most of the membrane exposed to serve as the fencing 14.

If the film 10, or an intended covering layer, is too soft to avoid deformation, an array of one or more spacers 22 may be provided to ensure that flanking channel 16 does not collapse. The spacers 22 can alternatively be provided on the covering layer. Controlled amounts of collapse may be desired to provide low pressure pumping and fluid recirculation in some embodiments.

Two ports 17 are provided on side 10a to serve as inlets or outlets for flanking channel 16. A third port 17 is a throughbore of the film 10, and as such connects with a CR defined as a recess 12 that is only in view from side 10b, and is accessible through the fencing 14. The patterning on side 10b shows a recess 12 in fluid communication with port 17. The recess 12 is roughly half a thickness of an intended bead bed, and a second instance of the film 10 is intended to be assembled with two covering layers, to produce a microfluidic chip for supporting an artificial biointerface. To assemble the two patterned films 10, the sides 10b face each other, but with the ports 17 on opposite sides of the recess 12. As such the total fluid path through recess 12 starts on side 10a of a first film 10, passes through first film 10 to a side 10b of the first film 10, crosses the recess 12 that is formed by the recesses of both the first film and second film (side 10b of each), then passes from side 10b to side 10a of the second film, to exit from the second film, side 10a. As a result both the flanking channel have two ports at opposite ends provided by the single film that defines the flanking channel on one side thereof, and the central region has one port defined on each film. Two covering plates are required, and ports are defined as is conventional, either on both sides of the assembled chip, at edges of the chips. An alternative arrangement uses more vias, and careful alignment of the films (which are differently patterned) to provide all ports on one side of the chip.

While the foregoing illustrated CRs, and parts thereof, with only two ports at opposite ends thereof, it will be appreciated that more than two ports may be provided if the CR is long enough that fluid pressures with entrained particles are difficult to control, or it is preferable to inject powders at different parts in the CR. In such cases, so that the ports may be used as either an inlet or an outlet, it may be preferable to use a removable porous plug within the port so that it can serve to extract fluid without the powder, or inject the powder.

FIGS. 2A,B are schematic flow charts of two processes for fabricating an artificial biointerface using a microfluidic structure using a film patterned in accordance with the present invention. Identical method steps are identified by like reference numerals and their explanations are not repeated. Both methods end with the production of a biointerface.

FIG. 2A begins with the supply of a microfluidic chip having a microfluidic chamber partitioned by fencing into 2 flanking channels (FC)s and a CR (step 30). The fencing is fluid permeable but retains beads, and therefore has a mean pore diameter of 0.8-100 μm, more preferably 1-20 μm, and most preferably 5-10 μm. The chip may be provided by forming a pattern on a film as described in FIG. 1 or its variants, and bonding a cover over at least the FCs and the CR, to enclose the chamber. The cover may have through-holes or re-sealable puncture-ready injection areas spaced to match the ports of the pattern on the film, in which case alignment is provided between the ports and these features. Alternatively an unstructured cover may be used and access to the ports may be provided by drilling through the cover.

One advantage of the use of a bead bed (a packing mostly consisting of rigid powders in which a particle density is sufficient so that at least ¾ of the particles contact at least 4 adjacent particles, and having at least 10% of the pores having a mean diameter matching mean diameter of the cells) as a scaffold for supporting cell culture over fabrics, functionalized plastics, and collagen mats, is the ability to distribute a variety of beads throughout the bead bed. The beads may vary by any feature that achieves the primary objectives of cell adhesion, cell alimentation, and cell growth, or secondary objectives of reporting cell activities, signaling, or excretions, or inducing changes to cell activities by emitting or selectively trapping signaling molecules or particles. At optional step 32, at least one powder is provided, the powder having a known size distribution, composition, morphology and packing density. The powder is divided into at least two segments, and each of the segments (or any mixture of the segments of different powders) is independently treated with, for example, by surface deposition of proteins, polymers, and other biochemical or organic products that are biocompatible and/or promote cellular adhesion, detection probes (protein, DNA, aptamers), stimulating agents (proteins, chemicals, drugs). For example, selective growth inhibitors, growth regulators, extra-cellular matrix (ECM), surface functionalizations for targeted reception of cell expressions, and timed-, delayed-, or triggered-release of a biomolecule or particle. The triggered-release treatment may result in release of the biomolecule or particle, such as a pharmaceutical formulation, in response to a change in pH, temperature, illumination, or a chemical reaction. By mixing the independently treated powder segments, with a carrier fluid (such as an aqueous buffer) a uniform and random distribution of the various functionalized particles may be provided. Any number of segments of any number of powders may be provided, as each powder may provide a different function or treatment. The treatment(s) may alter surface morphology (micro-/nano-structure) of the segments so that bed: better mimics physiological environments; or promotes cell adhesion and structuration (one, two or three dimensional arrangement of the cells to form a biowall).

Up to 25% of the particles of the mix may be non-rigid, (i.e. gelatinous, containing a non-trivial liquid or gaseous phase, or an elastomeric particle so soft as to deform under microfluidic shearing) particles that may provide other functionalities, without losing stability of the scaffold provided by the bead bed. The non-rigid particles in the mix (or alternatively the rigid particles of the bead bed) may include reporter, sensor, or delivery beads, particles, or objects (functional components). The bead bed offers the opportunity to reliably retain these functional components in situ with a required fixity, in an inter-region of the biointerface. The functional components may provide controlled release, selective release depending on pH, chemical, thermal, pressure, or like triggers sensed in situ within the inter-region, imparted externally, or with time. The functional components of the bead bed may interact with alimentation streams or waste products to emit signaling entities into a waste stream, or absorb or catalyse reactions, for example to promote, modulate or inhibit reporting, sensing or chemical release. Sensors are known for detecting: cell morphology changes, protein secretion and release, and genetic modifications. By monitoring waste products, feedback can be provided for varying alimentation of a biowall, inter-region, mucosa, or environment in accordance with an experimental objective.

At step 34, rigid powder is supplied into the CR via a port of the CR, in a liquid carrier. By blocking, and controlling pressures at, respective ports of the chamber, the liquid carrier can be extracted through the fencing or a filtered CR port to prevent loss of powders. The powder content is preferably controlled and metered to sufficiently fill the CR to form a packed bead bed. A fluid-dynamic resistance of the CR with the bead bed may be tested to confirm a density of the bead bed and to establish pressures required for alimentation.

At step 36 unattached live cells are supplied in a cell medium into at least one of the FCs. The medium may be extracted (at least in part) through a port of the opposite FR, or the filtered port of the CR to encourage deposition of the unattached live cells on a wall of the bead bed near the fencing. Alternatively the fluid may be supplied and settling of the cells onto the bead bed surface (scaffold) may be provided in time. Preferably at least one segment of the powders is treated with compounds selectively chosen to encourage cell attachment and growth. Alimentation of cells can be provided for by circulation of media and nutrients at step 38. The cells grow and form a biowall maintaining communication pathways and preferably native cell response. The alimentation may be provided using any of the ports of the chamber, and advantageously may include different media or nutrients on from the CR ports and the FC ports. Until cell attachment is established, or the biowall is formed to a certain degree, it may be preferable to maintain a slightly lower pressure within the CR than the FC, to ensure that the cells are drawn continuously towards the scaffold. Once a biowall is formed at one FC, the process may repeat to produce a second biowall on the opposite FC. Both biowalls may be produced concurrently.

It will be noted that the fencing serves not as a scaffold for cell growth, but rather as a boundary for a bead bed and therefore has minimal requirements other than to retain the beads. Once a cell culture is provided, the boundary may be largely irrelevant, or may still be required to ensure stability against fluid pressures. Thus a bioresorbable material could be used for forming the fencing.

Once the biowall(s) are formed, a variety of experiments can be performed, such as subjecting the biowall to a medicament, toxin, or other biomolecule or particle. The tests may be substantially non-intrusive, for example by sampling the circulating cell media, production of signaling molecules can be observed, without altering the cells. Naturally a feedback process can be provided with changes to the alimentation regime in response to detected changes in cell signaling. Furthermore the biowall may be imaged or otherwise examined to determine intracellular vs. extracellular components cells. In situ imaging may be possible with some transparent microfluidic materials. Finally the biowall or cells thereof may be lysed at the end of an experiment.

FIG. 2B is a flow chart showing a variant of the method of FIG. 2A, in which the bead bed is set in a different manner. Instead of starting with a complete microfluidic chip with the relevant chamber, a film with the relevant pattern is provided (step 40). At step 42, an optional mix of powders is provided. While this step may be identical to step 32, it may not be provided as a mix in a fluid carrier, despite the convenience of fluid carriage for preventing agglomeration and controlling flow of small amounts of powders. The powders may be pre-formed, or partially consolidated on a forming tool, such as a flat table or mold having a desired dimension, or pressing the powders through an extruder. The preformed bead bed may be filled with a liquid to avoid air pockets, and to increase adhesion and manipulability of the bead bed during placement. The pre-forming may be performed prior to, or after surface treatment, and the surface treatment may make the powders tacky enough to consolidate readily until exposed to a solvent, or more permanently adhesive. Whether placed in the CR as a preformed bed, or as a loose bed of powders, and whether the placement involves application of temperature, pressure or any setting agent, or removal of any liquid or other carrier, the bead bed is set within the CR at step 46. Subsequently the chamber is sealed with a cover by bonding the cover over at least the chamber, in a manner that allows access to the ports of the chamber (step 48). It will be noted that access to the CR prior to bonding the cover has some advantages and disadvantages depending on the specific bead bed and shape of the beads. In some embodiments, the bead bed may be formed or carried on a plastic film that becomes a cover, in which case step 46 is concurrent with step 48.

It should be noted that prior to step 36 of either process, a controlled supply of a treatment may be provided to functionalize a surface of the bead bed to a desired depth by providing immiscible fluids in the CR and FCs, such that varying pressure at the ports of the CR and FCs controls an interface between the two immiscible fluids. This may be particularly preferred for the method of FIG. 2A where the bead bed is not preformed and no prior opportunity is available for selectively treating the surface of the bead bed to which the cells will attach.

It will be noted that any variants of the patterned films 10 may equally be used to form a support for a biointerface according to the methods of FIG. 2A or 2B. It will be noted that in the examples of FIGS. 1,1A,1B, a depth of relief of the chamber 11 is generally less than 1 mm and this may make a pre-formed bead bed insert more difficult. The features 18 of FIG. 1A are unnecessary if the bead bed is inserted as a preform, and less likely to be used with the method of FIG. 2B. Furthermore the fencing 14 separating two parts of the divided bed as shown in FIG. 1A may be irrelevant if one or both of the sides is provided by a preform bead bed. If the CR has a non-uniform thickness, as shown in FIG. 1B, it may be particularly challenging to align a preform. The embodiment of FIG. 1 would require a preform in the form of a ribbon or rod of powder of rectangular cross-section. Sufficient mechanical cohesion and flexibility may be difficult to obtain, and moving the preform into position may be difficult in comparison with fluid injection. Thus the method of FIG. 2B is generally best suited to the embodiment of FIG. 1D.

While the illustrated embodiments and variants of FIG. 1-1C show planar microfluidic devices, it will be appreciated that some biointerfaces have inter-regions that are not ideally planar. Assuming elastomeric materials are used for at least one side of the microfluidic device (i.e. the patterned film or cover), and a high level of plasticity of the covering films, the planar pattern of the film can be adapted to a contour of a cover, or the cover may be deformed after the film is bonded thereto. If the pattern is applied to a non-planar (curved, bicurved, multiply curved) surface, an elastomeric cover can conform to the surface making a sealed microfluidic channel. The deformation of a planar microfluidic chip may be performed before or after the bead bed is put in place.

FIG. 3 is a schematic illustration of an artificial biointerface with cellular co-culture. Specifically FIG. 3 shows a chip (cover removed) formed with the patterning of FIG. 1, subjected to the methods of FIG. 2A or 2B. Cells 50a,b are grown on each side of the bead bed defining respective biowalls. A bead bed with 5 respectively functionalized powders 20a-e are shown. Each part may have particles coated to improve cell adhesion and growth, and a respective set of antibody-coated reporting beads.

### Examples

A wide variety of artificial biointerfaces can be produced on the biointerface substrate (the patterned film with the bead bed and a cover). For example (i) primary cerebral microvascular endothelial and astrocyte cells for blood-brain barrier, (ii) trophoblast and umbilical vein endothelial cells for placenta-fetal interface, (iii) alveolar epithelial cells and human pulmonary microvascular endothelial cells for lung alveolar-capillary interface, (iv) endothelial cell cytoplasmic cargo transport to stem cells, (v) any autocrine and paracrine cell-cell interactions for homeostasis, tissue repair and development etc.

Specifically this invention was demonstrated as a placenta-on-chip biointerface. FIG. 3 may further be regarded a schematic illustration of a placenta-on-chip biointerface for cellular co-culture. Cells 50a are placental trophoblast cells (JEG-3), and cells 50b are Human Umbilical Vein Endothelial Cells (HUVECs). Biomarkers associated with the respective bead bed parts include: β-hCG, GLUT1, IGF1/2, and VEGF, which are indicative of placental functionality and efficacy regarding nutrient transfer. The fifth part is reserved for experiment-specific monitoring. The top flanking channel (FC) will accordingly emulate a fetal flow and the bottom FC will emulate the mother flow.

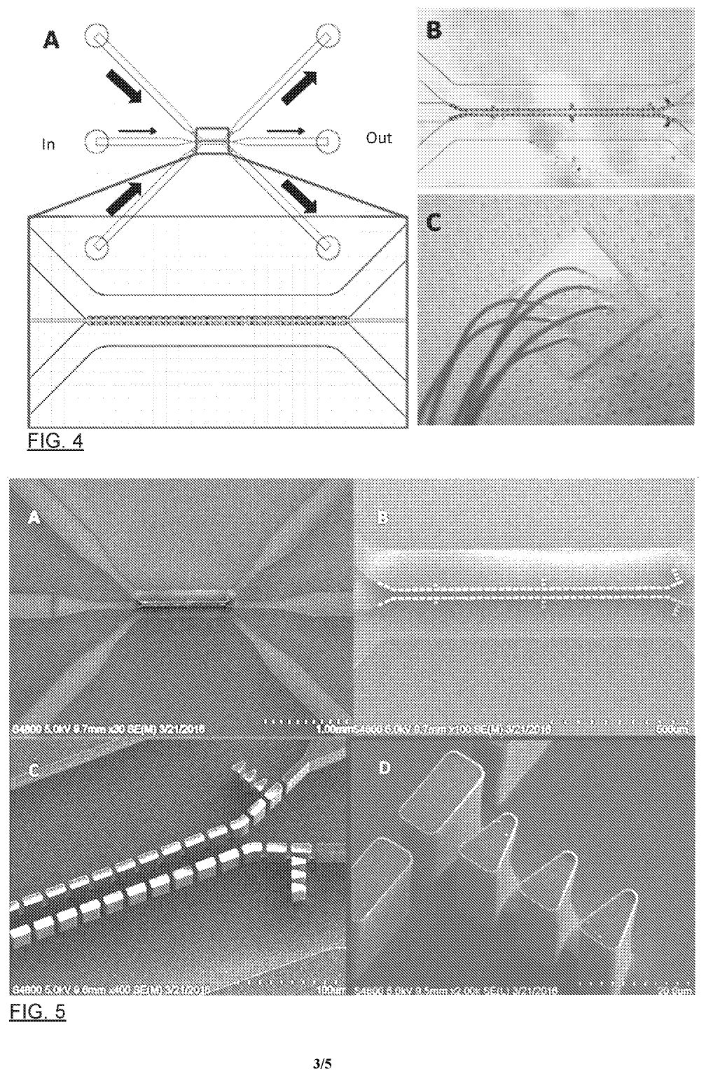

To make the patterned chip, the microfluidic patterned surface was produced and characterized to determine suitable flow parameters. The chamber, ports and interconnections are patterned on a Zeonor™ substrate, which is a rigid polycyclic olefin polymer. The cover is composed of oil-free Mediprene™, a thermoplastic elastomer with excellent adherence to a variety of substrates. FIG. 4 is a panel showing a microfluidic chip fabricated and used for demonstrating the present invention, to produce a placenta-on-chip biointerface. Specifically: FIG. 4A is a computer aided design (CAD) of the chip with an enlarged view of the biointerface in a divided chamber; FIG. 4B is an optical micrograph of the two FCs and the CR of the chamber; and FIG. 4C is a photograph of the chip with 6 ports thereof connected to 6 supply tubes. The chip is sided, with all inlets on the left, and all outlets on the right. The chamber has a length of about 1 mm, and a depth of about 50 μm.

FIG. 5 is a panel showing 4 enlargements of the chip: FIG. 5A showing the whole chip with a 1 mm scale (30× magnification); FIG. 5B showing the whole inter-region with a 500 μm scale (100× magnification); FIG. 5C showing half the inter-region with a 100 μm scale (400× magnification); and FIG. 5D showing flow control features of the flanking channel, and widening of the fencing with a 20 μm scale (2000× magnification). It will be noted that flow control features, best seen in FIG. 5B are distributed along the FCs to direct flow towards the fencing. The fencing as shown consists of rectangular pillars with a length of about 25 μm, a width of about 10 μm, and an inter pillar separation of about 5 μm. The ports are connected with 1/32″ PTFE tubing.

To assemble the microfluidic device, the patterned Zeonor substrate is ECM coated, and then sealed with an unpatterned film of Mediprene OF (the cover) into which holes are bored to permit fluid access to the ports. Once assembled, bead loading is performed to form a bead bed through the CR. Beads measuring 10 μm in diameter were loaded—either silica or polystyrene beads were used with no noticeable difference in terms of performance. Monodisperse beads were purchased from Corpuscular Inc. or Bangs Labs.

FIG. 6A is a system view of the fluid supply for: 1) cell seeding; 2) bead bed placement according to the method of FIG. 2A; 3) cell growth; and 4) experimentation. The system includes four computer-controlled syringe pump injectors, two reservoirs; and the chip. Specifically in this example a enMESIS™ Pump System (CETONI GmbH, Germany) was used to control delivery of nanoliter quantities of fluids to the ports of the chip by the tubing as shown in FIG. 4C. FIG. 6A schematically shows a fluorescence imaging apparatus useful for controlling operation and verifying steps, and FIG. 6B shows the imaging system with the chip and fluid supply system within a (temperature/CO₂ controlled) cell culture incubator of a fluorescence microscope.

Fluorescent visualization of rhodamine flow through the CR was used to determine satisfactory mass transfer conditions for ECM coating supplied through the CR (varied between 5-30 nL/min) while top and bottom FCs were held constant at 5 nL/min, and it was found that the best rate was around 10 nL/min.

Cell seeding of both populations is performed separately in order to better control cell density at the top and bottom compartments. First, placental JEG-3 cells in EMEM media (concentration of 1×10⁶ cells/mL) are loaded at the bottom channel inlet at a flow rate of 10 nL/min, while PBS and F-12 media are flown in the center and top channels, respectively, at 10 nL/min. Once enough cells are anchored to the pillars, flow is arrested and cells allowed to better adhere for 1 hour. Subsequently, the bottom channel flow is resumed and cell-free EMEM media is introduced at the inlet. The same procedure is then applied at the top channel when loading placental HUVEC cells. The cells are suspended in F-12 media at 1×10⁶ cells/mL and introduced through the top channel inlet at 10 nL/min. Meanwhile, PBS is flown through the center channel and EMEM through the bottom channel through inlet/outlet operation at 10 nL/min. Once enough HUVEC cells are loaded—akin to the JEG-3 cells in the opposite chamber—flow is resumed of cell-free F-12 media at the top channel and EMEM media at the bottom.

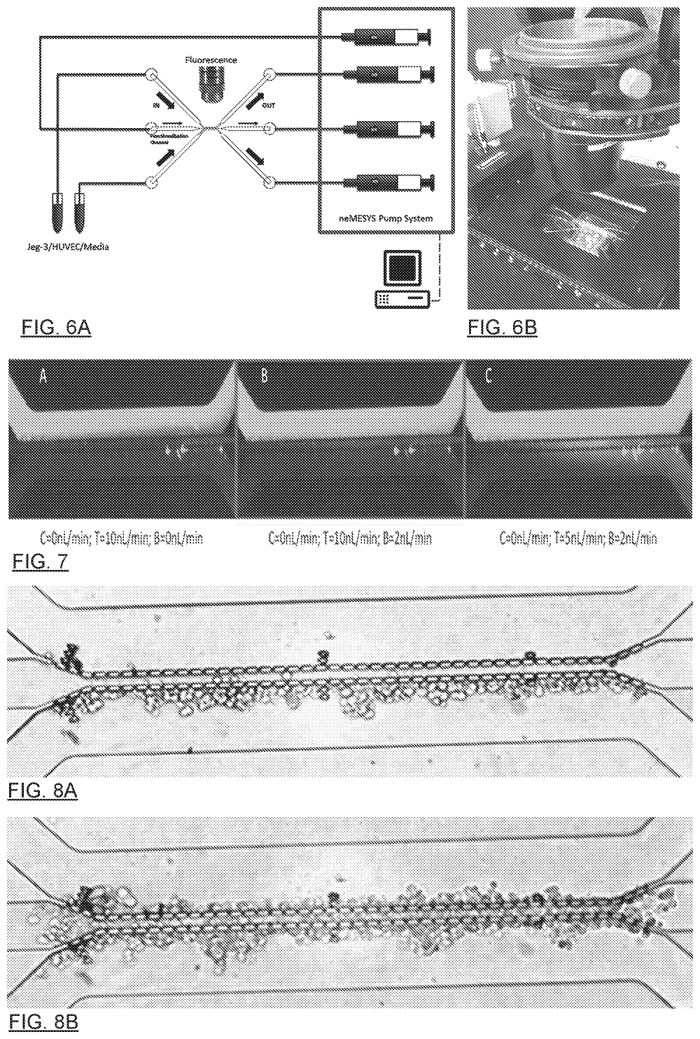

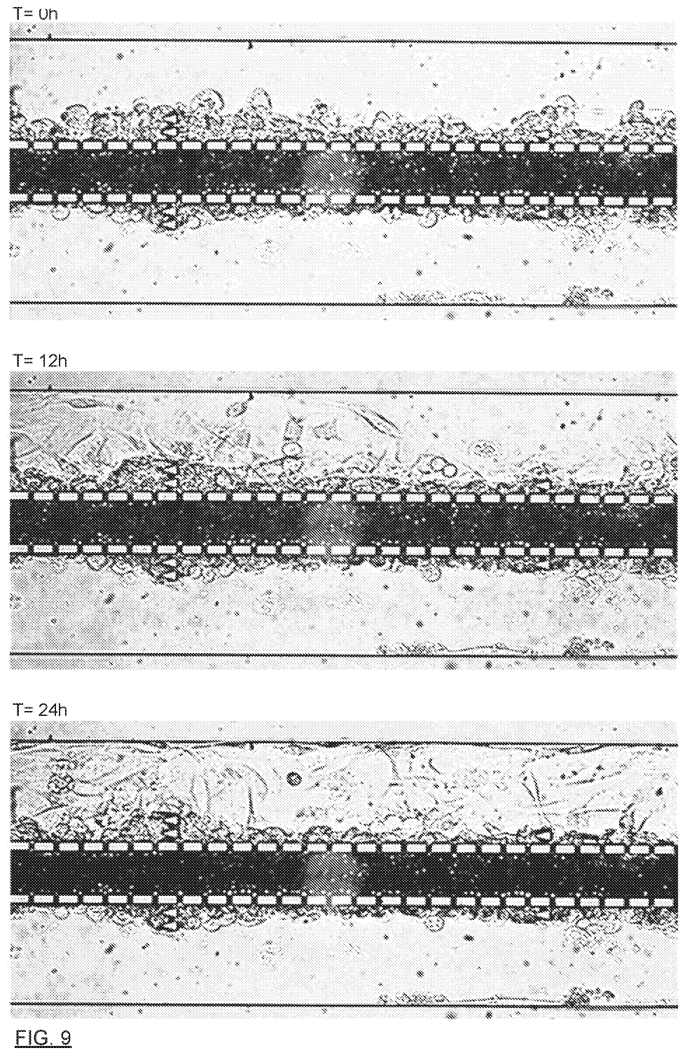

Similarly, as shown in FIG. 7, fluorescent visualization of rhodamine perfusion at various flow rates of the top and bottom FCs (keeping the flow through CR null) were examined to identify satisfactory mass transfer conditions to maintain a constant nutrient concentration across the inter-region. It was found that 10 nL/min through the top FC and 2 nL/min through the bottom FC were best.

Following cell seeding of JEG-3 and HUVEC, determined flow profiles are then implemented with 10 nL/min at the placental side and 2 nL/min at the fetal side, with the CR held static. Cell culture was then allowed to proceed in microscope CO₂ incubator and monitored by time-lapse microscopy for 24 hours. The resulting co-culture is clearly demonstrated in FIG. 9, with HUVECs spreading on the pillars to form a well-sealed monolayer—representative of endothelial physiology. JEG-3 cells maintained a more spherical morphology, while also forming a monolayer on their respective pillars opposite the HUVECs. It is interesting to note that the compacted bead bed also provided a cell culture surface for better cell anchoring, which both cell lines slightly traversed through the open pores at a distance of ˜5-10 μm from pillar structures. This results in a 3D co-culture interface that is intercalated with cells as opposed to traditional 2D flat surfaces culture. This in fact yields a more physiologically relevant interface with which to analyze cell-cell communication in response to biochemical stimuli.

FIGS. 8A,B are micrograph images of a two-step cell seeding process. FIG. 8A shows the JEG-3 cells loaded into bottom channel. FIG. 8B shows HUVEC cells loaded in top FC, permitting subsequent perfusion culture. The CR is shown empty as these experiments were performed to demonstrate that cell loading can be done effectively even in the absence of a packed bead bed. Note that unlike the previous figures, FIGS. 8A,B are shown with the flow directed from right to left.

FIG. 9 is a panel of three micrograph images showing biowall construction at 3 time steps: 0 h, 12 h, and 24 h. The time lapse images show the cellular co-culture on the bead bed scaffold. FIG. 9 shows a healthy, viable, culture throughout a 24 hours period.

The antibody-functionalized bead bed biointerface serves as the barrier as well as substrate for cell-cell signaling biomarker capture and detection. Applicant has subsequently experimented with embedding reporter coated particles within the bead bed. The beads were coated with anti-hCG antibody prior to loading into the device. Following cellular co-culture of Jeg-3 and HUVEC cells, the hCG released by Jeg-3 is captured on the bead surface during the course of the experiment. Following the co-culture period, the bead bed is subsequently perfused with fluorescently conjugated secondary anti-hCG antibody through the CR. The resulting fluorescent signal is correlated with hCG release into the cell-cell bead bed barrier in response to experimental stimuli.

The entire contents of the each of the following are incorporated by reference:
[1] Sams-Dodd F. Target-based drug discovery: is something wrong? Drug discovery today. 2005; 10:139-47.
[2] DiMasi J A, Feldman L, Seckler A, Wilson A. Trends in risks associated with new drug development: success rates for investigational drugs. Clinical pharmacology and therapeutics. 2010; 87:272-7.
[3] DiMasi J A, Hansen R W, Grabowski H G. The price of innovation: new estimates of drug development costs. Journal of health economics. 2003; 22:151-85.
[4] Sangeeta N Bhatia, Donald E Ingber, Nature Biotechnology 32, 760-772 (2014) doi:10.1038/nbt.2989
[5] Sung J H, Shuler M L. Microtechnology for mimicking in vivo tissue environment. Ann Biomed Eng. 2012; 40:1289-300.
[6] Moraes C, Mehta G, Lesher-Perez S C, Takayama S. Organs-on-a-chip: a focus on compartmentalized microdevices. Ann Biomed Eng. 2012; 40:1211-27.
[7] Huh D, Torisawa Y S, Hamilton G A, Kim H J, Ingber D E. Microengineered physiological biomimicry: organs-on-chips. Lab Chip. 2012; 12:2156-64.
[8] Price G M, Wong K H, Truslow J G, Leung A D, Acharya C, Tien J. Effect of mechanical factors on the function of engineered human blood microvessels in microfluidic collagen gels. Biomaterials. 2010; 31:6182-9.
[9] Esch M B, Sung J H, Yang J, Yu C, Yu J, March J C, et al. On chip porous polymer membranes for integration of gastrointestinal tract epithelium with microfluidic ‘body-on-a-chip’ devices. Biomed Microdevices. 2012; 14:895-906.
[10] Sung J H, Yu J, Luo D, Shuler M L, March J C. Microscale 3-D hydrogel scaffold for biomimetic gastrointestinal (GI) tract model. Lab Chip. 2011; 11:389-92.
[11] Prot J M, Aninat C, Griscom L, Razan F, Brochot C, Guillouzo C G, et al. Improvement of HepG2/C3a cell functions in a microfluidic biochip. Biotechnol Bioeng. 2011; 108:1704-15.
[12] Domansky K, Inman W, Serdy J, Dash A, Lim M H, Griffith LG. Perfused multiwell plate for 3D liver tissue engineering. Lab Chip. 2010; 10:51-8.
[13] Khetani S R, Bhatia S N. Microscale culture of human liver cells for drug development. Nat Biotechnol. 2008; 26:120-6.
[14] Huh D, Matthews B D, Mammoto A, Montoya-Zavala M, Hsin H Y, Ingber D E. Reconstituting organ-level lung functions on a chip. Science. 2010; 328:1662-8.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method for modelling a biointerface, the method comprising:
providing a patterned microfluidic chip, the chip having:
a chamber divided into a central region and a first and second flanking channels that flank the central region, the division provided by a fluid-permeable fencing; and
at least 3 microfluidic ports, including at least two ports at opposite ends of the chamber, and at least one ports in each of one of the central region and the two flanking channels;
localizing a porous packing of rigid beads within the central region to define a bead bed, the beads having a mean size, between 2 and 300 μm, sufficient for the fencing to retain the beads while fluid permeates the fencing; and
growing a biowall on at least one segment of the fencing separating the central region from one flanking channel, the biowall formed at least in part by live cells cultured on the beads, by alimenting cells through the pairs of microfluid ports.

2. The method of claim 1 wherein each of one of the central region and the two flanking channels has at least two ports located at opposite ends of the chamber.

3. The method of claim 1 wherein the beads are composed of a polymer, glass, metal, or ceramic.

4. (canceled)

5. The method of claim 1 wherein: at least a first fraction of the beads are treated: to improve cell adhesion or growth; to selectively bind to a target molecule or particle; to report binding to a target molecule or particle; to selectively release a molecule or particle; to selectively bind, report binding, or selectively release a target molecule or particle in response to optical, thermal, electrical, magnetic, chemical, or mechanical stimulation; or for time-dependent selectively binding, report of binding, or selectively release of a target molecule or particle; or the packing of rigid beads comprises at most 25% of non-rigid beads, particles or objects.

6. The method of claim 1 wherein the fencing has through-holes from the flanking channel side to the central region that are smaller than a mean diameter of the smallest 10% of the beads; or the fencing has a 1D, 2D or 3D curvature for delimiting the packing of beads to define a shape suited to mimicking a geometry of a natural tissue.

7. (canceled)

8. (canceled)

9. The method of claim 1 further comprising: coating the chamber with a cell adhesion promoting coating; localizing the porous packing by introducing a mixture of the beads into the chamber; seeding at least one cell culture through at least one of the flanking channels; and incubating while alimenting the at least one cell culture.

10. The method of claim 9 wherein introducing the mixture of the beads into the chamber comprises:
injecting the bead mixture in a liquid carrier through one of the ports of the central region while extracting fluid at one of the other ports;
placing a pressed mixture of the beads into the central region with a cover of the microfluidic chip removed; or
introducing at least two phases into respective parts of the central region, each phase having a different constituency in that terms of at least one of: a mean size, mean shape, surface texture, functionalization, composition, or coating of the beads, or fractional populations of a mixture of such beads along with any non-rigid beads, particles or objects.

11. (canceled)

12. (canceled)

13. The method of claim 10 wherein the respective parts of the central region partition the central region in lines perpendicular to a flow between a pair of ports of the central region.

14. (canceled)

15. The method of claim 1 wherein the central region is an elongated flow path through the patterned microfluidic chip having a length between two opposing ports that is at least 2 orders of magnitude greater than an etch depth dimension of the central region, and at least twice that of a width of the central region.

16. A kit for producing an artificial biointerface, the kit comprising:
a patterned microfluidic surface, the pattern defining a chamber divided into a central region and a first and second flanking channels that flank the central region, the division provided by a fluid-permeable fencing;
a source of rigid beads adapted to form a bead bed within the central region, the beads having a mean size between 2 and 300 μm, sufficient for the fencing to retain the beads while fluid permeates the fencing; and
a cover for the microfluidic surface, the cover dimensioned for enclosing the chamber and adapted to seal the chamber from ambience;
wherein at least one of the patterned microfluidic surface, and cover provide at least 3 microfluidic ports, including two ports at opposite ends of the chamber, and at least one port in each of the central region and the two flanking channels; and
the fencing has gaps having a mean pore equivalent diameter, and the mean size of the rigid beads is at least 5% larger than the mean pore equivalent diameter.

17. The kit of claim 16 wherein each of the central region and the two flanking channels has two ports located at opposite ends of the chamber.

18. The kit of claim 16 wherein the beads are composed of a polymer, silica, metal, or ceramic.

19. (canceled)

20. The kit of claim 16 wherein at least a first fraction of the beads are treated: to improve cell adhesion or growth; to selectively bind to a target molecule or particle; to report binding to a target molecule or particle; or to selectively release a molecule or particle.

21. The kit of claim 20 wherein said target molecule or particle is bound, reported bound, or released in response to optical, thermal, electrical, magnetic, chemical, or mechanical stimulation.

22. (canceled)

23. The kit of claim 16 wherein the fencing has: through-holes from the flanking channel side to the central region that are smaller than a mean diameter of the smallest 10% of the beads; or a 1D, 2D or 3D curvature for delimiting the packing of beads to define a shape suited to mimicking a geometry of a natural tissue.

24. (canceled)

25. The kit of claim 16 wherein the packing of rigid beads comprises: at most 25% of non-rigid beads, particles or objects; or at least two phases, each phase having a different constituency in that terms of at least one of: a mean size, mean shape, surface texture, functionalization, composition, or coating of the beads, or fractional populations of a mixture of such beads along with any non-rigid beads, particles or objects.

26. (canceled)

27. The kit according to claim 16, wherein the kit is assembled with the bead bed formed within the central region and the cover enclosing and sealing the chamber.

28. The kit according to claim 27 with a biowall formed located along a segment of the fencing separating the central region from one flanking channel, the biowall formed at least in part by live cells cultured on the beads, the biowall being alimented by the microfluidic ports.

29. An artificial biointerface comprising:
a microfluidic chamber divided into a central region and a first and second flanking channels that flank the central region, the division provided by a fencing;
the central region filled with a porous packing comprising rigid beads, the beads having a mean size between 2 and 300 μm, sufficient for the fencing to retain the beads while fluid permeates the fencing;
a biowall located along a segment of the fencing separating the central region from one flanking channel, the biowall formed at least in part by live cells cultured on the beads; and
at least 3 microfluidic fluid paths, each of the paths extending across one of the two flanking channels, and the central region, for supplying and extracting fluid from the respective flanking channel or central region.

30. (canceled)

31. (canceled)

32. (canceled)

33. The artificial biointerface of claim 29 wherein: at least a first fraction of the beads are treated: to improve cell adhesion or growth; to selectively bind to a target molecule or particle; to report binding to a target molecule or particle; or to selectively release a molecule or particle; the packing of rigid beads comprises at most 25% of non-rigid beads, particles or objects; or the packing of rigid beads comprises at least two phases, each phase having a different constituency in that terms of at least one of: a mean size, mean shape, surface texture, functionalization, composition, or coating of the beads, or fractional populations of a mixture of such beads along with any non-rigid beads, particles or objects.

34. (canceled)

35. (canceled)

36. The artificial biointerface of claim 29 wherein the fencing has through-holes from the flanking channel side to the central region that are smaller than a mean diameter of the smallest 10% of the beads; or the fencing has a 1D, 2D or 3D curvature for delimiting the packing of beads to define a shape suited to mimicking a geometry of a natural tissue.

37. (canceled)

38. (canceled)

39. (canceled)
